(19) United States
(12) Patent Application Publication
(10) Pub. No.: US 2010/0152748 A1
(43) Pub. Date: Jun. 17, 2010

(54) Devices, Systems, and Methods Providing Body Lumen Access

(75) Inventors: Abraham Penner, Tel Aviv (IL); Lone Wolinsky, Ramat Gan (IL); Alon Ben-Yosef, Ramot Manasha (IL)

Correspondence Address: SUTHERLAND ASBILL & BRENNAN LLP, 999 PEACHTREE STREET, N.E., ATLANTA, GA, 30309, US

(73) Assignee: E-PACING, INC., Wilmington, DE (US)

(21) Appl. No.: 12/637,081

(22) Filed: Dec. 14, 2009

Related U.S. Application Data
(60) Provisional application No. 61/122,217, filed on Dec. 12, 2008.

(51) Int. Cl. A61B 17/00; A61M 29/00

(52) U.S. Cl. 606/129; 606/191

(57) ABSTRACT

Devices and methods are provided which include access port devices for implantation through a tissue wall of a lumen of patient's body and for containing electrical leads therein. An implantable port device includes a body with a first end having a first opening and an opposed second end having a second opening, and a channel extending from between and operably connecting the first opening and the second opening; a first retaining member extending radially from the first end of the body; and a second retaining member spaced apart from the first retaining member, the second retaining member being closer than the first retaining member to the second end of the body, and extending radially from the second end of the body. The first retaining member and the second retaining member are configured to cooperatively engage opposing sides of the tissue wall about edges of an aperture through the tissue wall to secure the body within the aperture.

### CROSS REFERENCE TO RELATED APPLICATIONS

This application claims priority to U.S. Provisional Application Ser. No. 61/122,217, entitled “Devices, Systems, and Methods Providing Respiratory Tract Access,” filed on Dec. 12, 2008, which is incorporated herein by reference.

### BACKGROUND

This disclosure relates generally to the field of medical systems and treatment methods, and more particularly to devices, systems, and methods providing body lumen access.

Certain cardiac deficiencies, such as cardiac arrhythmias including bradycardia and tachycardia can be treated by pacemakers or by implantable cardioverter-defibrillators. A pacemaker is an electronic device that may pace or regulate the beating of a patient's heart by delivering precisely timed electrical stimulation to specific areas of the heart, depending upon the condition being treated. For example, bradycardia, a condition in which a patient's heart rate is slowed, or tachycardia, a condition in which a patient's heart rate is too fast, may be treated by performing cardiac pacing. As used herein, the term “pacemaker” may refer to any cardiac rhythm management device that is operable to perform cardiac pacing, regardless of any other functions it may perform.

Cardiac stimulation devices may include implantable cardioverter-defibrillators, which may also be interchangeably referred to herein as “cardioverters,” “defibrillators,” or “ICDs.” Implantable cardioverter-defibrillators perform functions similar to pacemakers by delivering electrical pulses. However, ICDs are often used to treat sudden cardiac arrhythmias, such as atrial or ventricular fibrillation or ventricular tachycardia. Most ICDs operate by monitoring the rate and/or rhythm of a patient's heart and deliver electrical pulses and/or electrical shocks when abnormalities are detected. For example, some ICDs may be configured to deliver electrical shocks, while other ICDs may be configured to first deliver lower power electrical pulses to pace the heart prior to delivering electrical shocks.

In order to electrically stimulate the heart, electrodes typically are positioned and fixed close to the required stimulation site. Some conventional cardiac stimulation techniques deploy a transvenous electrode by transvenous catheterization to the right atrium or the right ventricle, or to both, for performing dual chambers pacing. Other conventional cardiac stimulation devices include epicardial electrodes deployed to the epicardium at various locations.

In addition to generating and delivering electrical stimulation to a patient's heart, cardiac treatment devices can be configured to measure various physiological parameters to aid in detecting and treating cardiac deficiencies. For example, sensing the heart's electrical activity allows detecting many cardiac deficiencies, including, but not limited to, bradycardia, tachycardia, atrial fibrillation, and myocardial infarction. Additionally, synchronization (and/or asynchronization) may be detected between relative heart chambers using cardiac devices, including detecting the delay between right atrium and right ventricle (“A-V delay”) and the delay between the right and left ventricles (“V-V delay”), which may assist in detecting and treating heart deficiencies. Furthermore, some conventional cardiac treatment devices can measure electrical impedance proximate the heart to detect fluid congestion in the lungs, which may indicate congestive heart failure. Conventional cardiac treatment devices may further include other sensors, such as accelerometers, flow monitors, and oxygen sensors, for example, for measuring other conditions related to a patient's cardiac performance.

Such conventional cardiac stimulation and sensing devices and associated detection and treatment techniques can require complex and highly invasive implantation procedures for electrode and pulse generator placement, increasing the risk of infection and other complications. Electrical leads carrying electrodes or other sensors to the treatment site are also subjected to mechanical fatigue as a result of the conventional deployment techniques and paths that are often dictated by vasculature or cardiac anatomy, causing lead or electrode failure.

Accordingly, it is desirable to provide devices, systems, and methods which provide access to body lumens, such as an airway or gastrointestinal tract.

### SUMMARY

Devices and methods described herein provide access to a body lumen using an access port device for implantation through a patient's tissue wall and for containing electrical leads therein. According to one aspect, an implantable port device for providing access through a tissue wall of a lumen of a patient's body is provided. The implantable port device includes a body with a first end having a first opening and an opposed second end having a second opening, and a channel extending from between and operably connecting the first opening and the second opening. In one embodiment, the device further includes a first retaining member extending radially from the first end of the body and a second retaining member spaced apart from the first retaining member, the second retaining member being closer than the first retaining member to the second end of the body, and extending radially from the second end of the body. In one embodiment, the first retaining member and the second retaining member are configured to cooperatively engage opposing sides of the tissue wall about edges of an aperture through the tissue wall to secure the body within the aperture.

According to another aspect, a guidewire and a removable dilator are further provided. The guidewire is adapted to penetrate the tissue wall to form an aperture therein. The dilator is adapted to slide over the guidewire and to expand the aperture when inserted therethrough.

According to another aspect, a method of implanting an access port device in a patient in need thereof is provided. In one embodiment, the method includes penetrating a lumenal tissue wall using a guidewire, forming an aperture therein; attaching an access port device to the guidewire; pulling the guidewire through the tissue wall in a manner effective to pull the access port into a position within the aperture of the tissue wall; detaching the guidewire from the access port device; and removing the guidewire from the lumen of the lumenal tissue wall.

According to yet another aspect, a kit for implanting an access port device in a tissue wall of a lumen of a patient's body is provided. The kit includes an access port device, a guidewire, and a dilator. The access port device includes a body with a first end having a first opening and an opposed second end having a second opening, and a channel extending from between and operably connecting the first opening and the second opening. The access port device can further include a first retaining member extending radially from the first end of the body and a second retaining member spaced apart from the first retaining member, the second retaining member being closer than the first retaining member to the second end of the body, and extending radially from the second end of the body. The guide wire is configured for penetrating the tissue wall and forming an aperture therein, and/or for inserting the access port device through the aperture formed in the tissue wall. The dilator is configured for enlarging the aperture formed in the tissue wall.

### BRIEF DESCRIPTION OF THE DRAWINGS

FIGS. 1A-1B are diagrams showing placement of a cardiac device, according example embodiments.

FIGS. 2A-2E are cross-sectional views of various embodiments of a respiratory tract access port.

FIG. 3 is a cross-sectional view of a respiratory tract access port and delivery device, according to one embodiment.

FIG. 4 is a cross-sectional view of a respiratory tract access port and delivery device, according to another embodiment.

FIG. 5 is a flowchart of a method for implanting a respiratory tract access port, according to one embodiment.

FIG. 6 is a cross-sectional view of an apparatus to facilitate deployment of an implantable device, according to one embodiment.

FIG. 7 is a diagram of an endoscopic apparatus in use to facilitate deployment of an implantable device, according to one embodiment.

### DETAILED DESCRIPTION

The human anatomy beneficially provides access to electrode implantation sites within the patient's airway that are in close proximity to areas of the heart, and thus allows for alternative implantation devices and methods for electrically stimulating the heart and/or for sensing cardiac activity. Stimulation and/or sensing electrodes and/or wireless transmitting leads can be implanted within a patient's airway using minimally or non-invasive techniques, thus avoiding the complex, higher-risk procedures associated with traditional implantation and stimulation techniques. In some cases, a pulse generator or other controller for operably communicating with the electrodes may be implanted subcutaneously, requiring electrical leads to pass through the patient's airway. Accordingly, an access port has been developed to provide access to a body lumen and for containing electrical leads therein. An access port according to the various embodiments described herein advantageously can be positionable and fixable within an airway wall, and used to facilitate deploying and containing electrical leads passing through the airway wall, for example from a subcutaneously implanted pulse generator to within the patient's airway. In another embodiment, an access port is positionable and fixable within any lumenal tissue wall other than an airway wall, such as the wall of a patient's digestive tract, for deploying and containing electrical leads therein. As used herein, the term “lumenal tissue wall” generally refers to any tissue wall of a body lumen.

A detailed description of an implantable system for the stimulation of the heart, phrenic nerve, or other tissue structures accessible via a patient's airway is included in U.S. patent application Ser. No. 12/128,489, entitled “Implantable Devices and Methods for Stimulation of Cardiac or Other Tissues,” filed on May 28, 2008, which is incorporated herein by reference in its entirety. A detailed description of an implantable system for the stimulation of the heart, phrenic nerve, or other tissue structures accessible via a patient's gastrointestinal system is included in U.S. patent application Ser. No. 12/136,812, entitled “Implantable Devices and Methods for Stimulation of Cardiac or Other Tissues,” filed on Jun. 11, 2008, which is incorporated herein by reference in its entirety. A detailed description of an implantable system for the stimulation of the heart, phrenic nerve, or other tissue structures accessible via a patient's gastrointestinal system is included in U.S. patent application Ser. No. 12/578,370, entitled “Devices and Methods for Electrical Stimulation of the Diaphragm and Nerves,” filed on Oct. 13, 2009, which is incorporated herein by reference in its entirety.

The devices and associated methods described herein facilitate deployment and operation of implantable cardiac, diaphragm, and/or nerve stimulation devices that include passing electrical leads through a patient's tissue wall (e.g., airway or digestive tract) and into a body lumen defined by the tissue wall. More specifically, in one embodiment an access port is provided to transverse a patient's airway (or other body lumen) and for providing access therethrough. The access port may be used to deploy and contain one or more electrical leads between a patient's thoracic cavity, or other subcutaneous location, and the patient's airway.

In some embodiments, the access port includes features that facilitate acute fixation and/or chronic fixation to an airway wall and/or that seal the thoracic cavity from airway environment. In one embodiment, these features include a first retaining member and a second retaining member, each extending radially from the body of the access port and positioned to retain the airway wall therebetween. In one embodiment, the first retaining member can be formed in a barb or other conical configuration to facilitate insertion through the airway wall. In one embodiment, the access port also includes an inner seal for containing and sealing the electrical lead. Various features of the access port can be formed from rigid materials to provide structural rigidity to the aperture formed in the patient's airway, while other features can be formed from semi-rigid or pliable materials to enable deformation thereof to conform to, and provide a seal with, the aperture formed in the airway and/ the electrical leads or other components contained by the access port. The access port may be deployed from a position external to a patient's airway, for example, from the patient's thoracic cavity, or from a position within the patient's airway.

The terms “airway” and “respiratory tract” are used interchangeably and may refer to the bronchi and/or the trachea. The terms “bronchus,” “bronchi,” and “bronchial tree” as used herein may refer generally to any of the individual components of the bronchi, including the primary bronchi, the secondary bronchi, the tertiary bronchi, and/or the bronchioles branching therefrom.

The terms “access port,” “respiratory tract access port,” and “cannula” are used interchangeably and may refer generally to any suitable device providing access through a tissue by at least one channel extending therethrough.

The terms “lead,” “electrical lead,” and “stimulation lead” are used interchangeably and may generally refer to any conductor for conducting electrical current between a pulse generator (or any other signal generator and/or receiver) and an electrode. In some embodiments, a lead may be coated with an insulating material, such as a polymer insulator like silicone, polyurethane, perflourocarbon, ePTFE, or any combination thereof. In one embodiment, one or more leads may each have one or more conductors allowing for sensing, pacing, defibrillation, or any other stimulation using a single lead. An electrode may be positioned in proximity to the distal end of the electrical lead, and/or at a position proximal to the electrical lead's distal end, such as when a lead includes two or more electrodes. For example, proximally positioned electrodes may serve a similar purpose as conventional leads that implant electrodes in a patient's vena cava. Leads may have many suitable configurations, including, but not limited to, true bipolar, single-coil, dual-coil, active fixation, or passive fixation leads. In some embodiments, the lead length may vary depending upon its intended application and/or placement. Leads may also optionally include a drug elution means, such as steroid elution to mitigate scar tissue formation. Drug elution means may be incorporated at or proximate the lead's distal tip, or at any other point along the lead length.

In other aspects, the systems, devices, and methods described herein can be used with devices that provide stimulation and/or sensing of any tissue accessible via a patient's airway, and are not limited to cardiac stimulation and/or sensing. For example, the phrenic nerve may be stimulated to activate the diaphragm, or the diaphragm may be directly stimulated, to provide therapy to patient's suffering from respiratory ailments.

While the embodiments described herein illustrate an access port used to transverse a patient's airway, the same or similar embodiments can be used to transverse a body lumen and tissue wall other than a patient's airway, such as a patient's gastrointestinal tract. Accordingly, the dimensions described herein may be altered accordingly to provide suitable adaptation for different uses.

Like numbers refer to like elements throughout the following description.

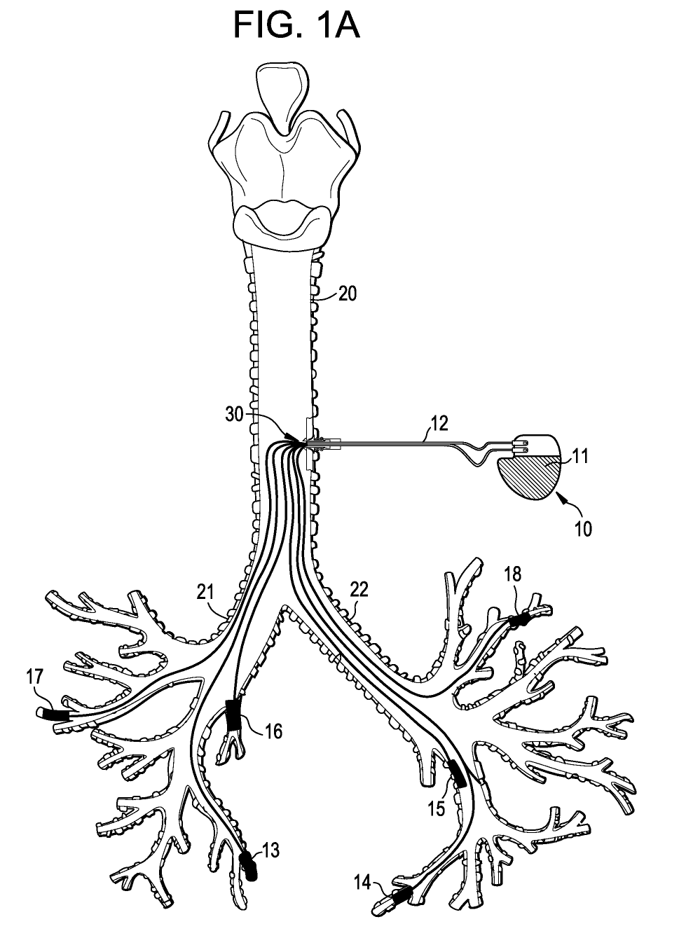

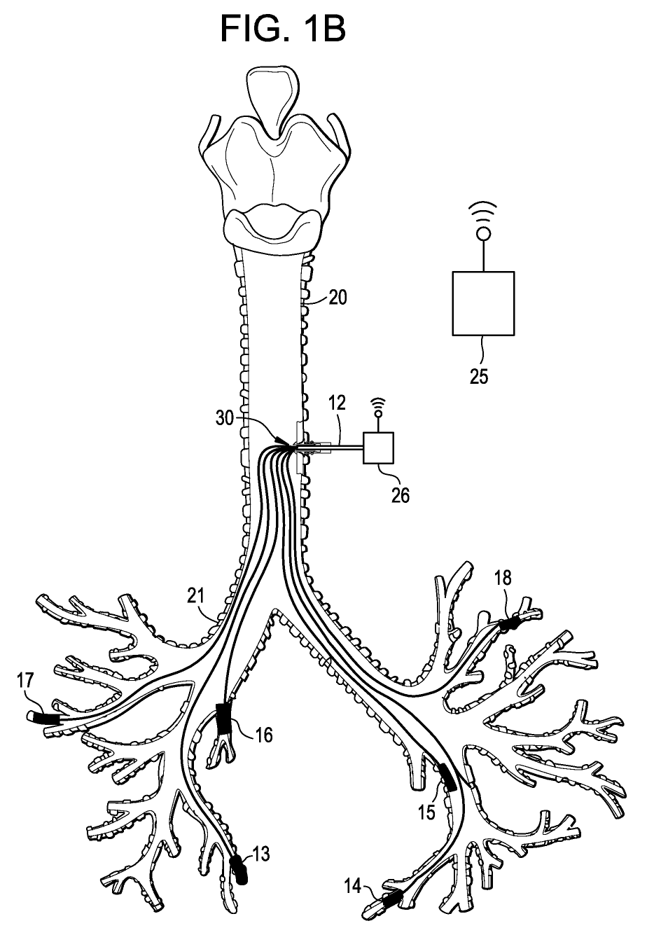

With reference to the Figures, FIG. 1A depicts a stimulation system, such as for performing cardiac stimulation, according to one embodiment. The implantable stimulation system includes a controller housing 10 that includes a pulse generator 11, at least one electrical lead 12, and a respiratory tract access port 30. In one example, the controller housing 10 is surgically implanted subcutaneously, such as approximately in a patient's pectoral region, and a subcutaneous tunnel is formed between the controller housing 10 and a position on the patient's airway, such as a position on the trachea 20 or primary bronchus 21, 22. At the interface with the airway, the airway wall is punctured and an aperture is created therein. A respiratory tract access port 30 is implanted into the aperture to provide a passageway for the electrical leads 12 to pass through the airway wall and into the airway. At least one electrode, such as one or more electrodes 13, 14, 15, 16, 17, 18, is carried by one or more electrical leads 12 and is positioned at a desired stimulation site within the patient's airway, such as within the secondary or tertiary bronchi or within the bronchioles.

According to one embodiment, a single electrical lead 12 is passed through the respiratory tract access port 30 and branches into multiple leads, each including at least one electrode positionable within the patient's airway. In another embodiment, however, multiple electrical leads 12 are passed through the respiratory tract access port 30, each including at least one electrode positionable within the patient's airway. In yet another embodiment, a single electrical lead 12 is passed through the respiratory tract access port 30, whereby the single electrical lead 12 includes multiple electrodes positionable within the patient's airway.

FIG. 1B depicts another stimulation system, according to another embodiment. One or more electrical leads 12, each carrying one or more electrodes 13, 14, 15, 16, 17, 18, are positionable within a patient's airway. Each electrical lead 12 is connected to a relay unit 26 implanted subcutaneously and operable to electrically communicate (e.g., wirelessly or wired) with a non-implanted pulse generator or other controller 25 positioned outside of the patient's body. In one embodiment, the relay unit 26 is implanted outside of the airway, such as within the thoracic cavity or within the patient's gastrointestinal tract. According to another embodiment, the relay unit 26 is implanted within the airway, such as within the trachea 20 or primary bronchus 21, 22.

In embodiments that include a relay unit 26 operable to wirelessly communicate with a controller 25, any number of means for performing wireless communications may be employed. For example, electrical signals (to direct stimulation and/or sensing) may be wirelessly communicated from the controller 25 to the relay unit 26 by electromagnetic induction, radio frequency, ultrasonic, infrared, or any other known wireless communication protocol. In one example, the relay unit 26 may include a wireless transmitter and receiver operable to communicate wirelessly through any of the aforementioned or other suitable wireless protocol. The relay unit 26 may further include electronic circuitry, a power source (if an active device), hardware, and/or software for receiving and transmitting wireless communications from and to the controller 25, and for generating electrical stimulation pulses or performing sensing functions via the one or more electrical leads and electrodes. In another embodiment, the relay unit is a passive device that does not include a power source, but the energy required to generate the stimulation signals and/or to perform the sensing operations is transmitted from the controller 25 using passive wireless communications (e.g., passive induction or passive RF communications). Accordingly, in a passive configuration, the relay unit 26 may include electronic circuitry for receiving the energizing signal (e.g., via induction, RF, etc.), optionally decoding the information transmitted thereby, and for generating electrical signals, such as for stimulation or sensing. Thus, the electronic circuitry of a relay unit 26 can generate the stimulation energy (e.g., through capacitive charging and discharging), instead of receiving it from the controller 25.

In another embodiment, one or more electrical leads 12, carrying one or more electrodes 13, 14, 15, 16, 17, 18 positionable within a patient's airway, are connected directly to a non-implanted controller 25 positioned outside of the patient's body. The one or more electrical leads 12 may be configured to pass from a subcutaneous location to the patient's airway through a respiratory tract access port 30, as described herein. The electrical leads 12 may pass from a subcutaneous location to the non-implanted controller 25 through one or more incisions, via a catheter, cannula, or any other suitable means. In another embodiment, the one or more electrical leads 12 are connected to subcutaneously implanted relay unit 26 that includes one or more electrical connectors exiting from the patient's body (e.g., via a catheter, cannula, etc.). A non-implanted controller 25 of this embodiment is configured to connect to the one or more electrical connectors exiting the patient's body.

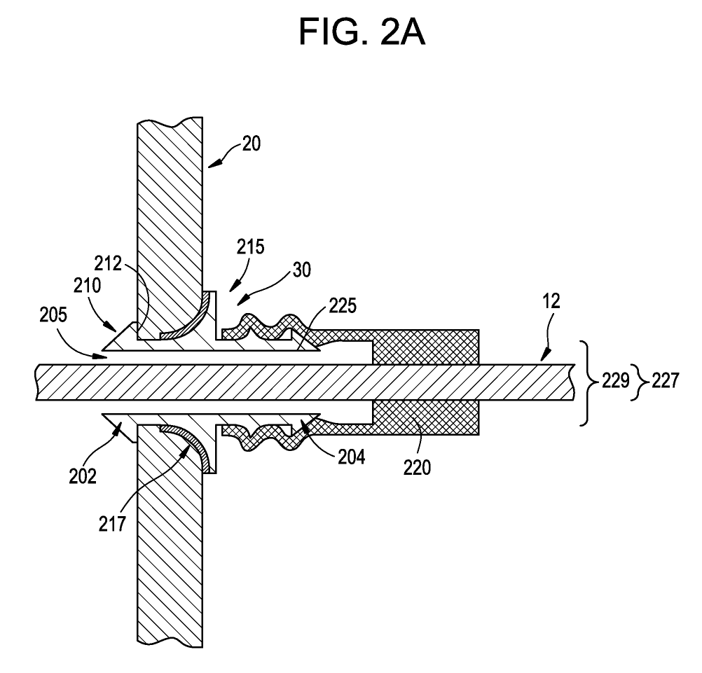

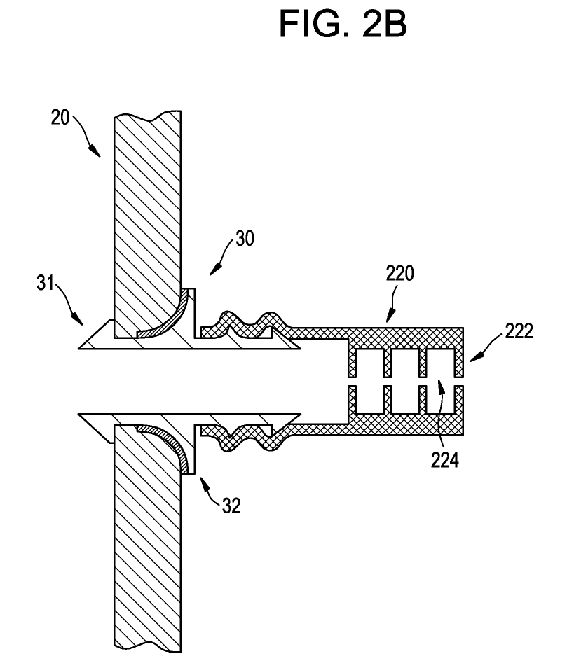

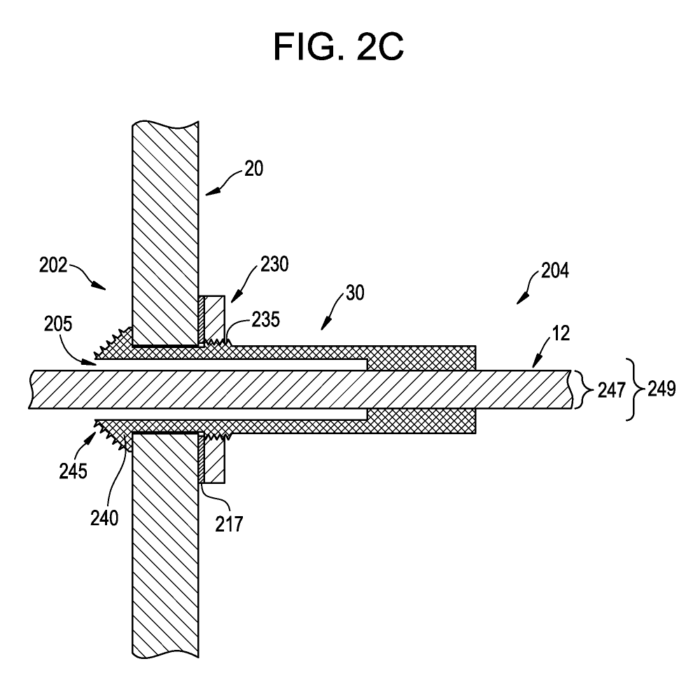

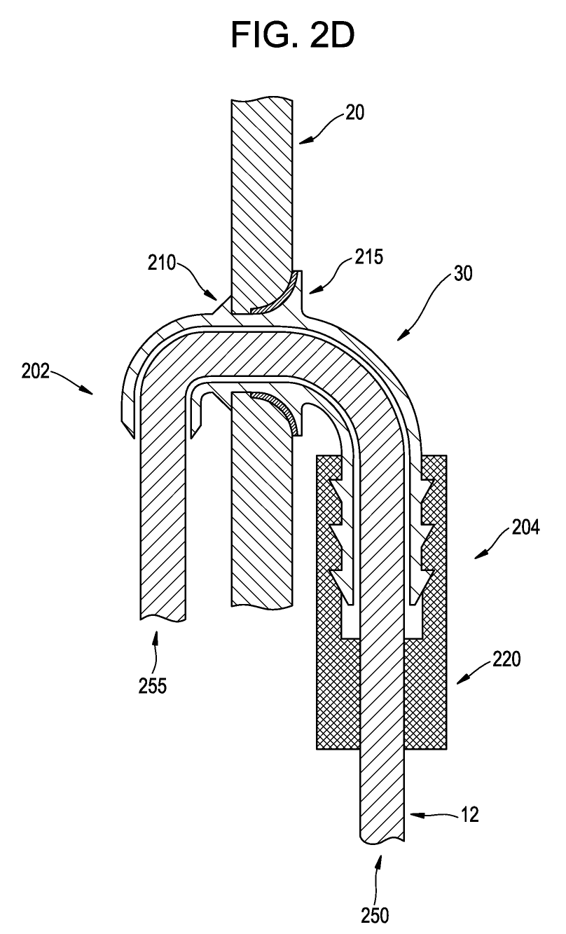

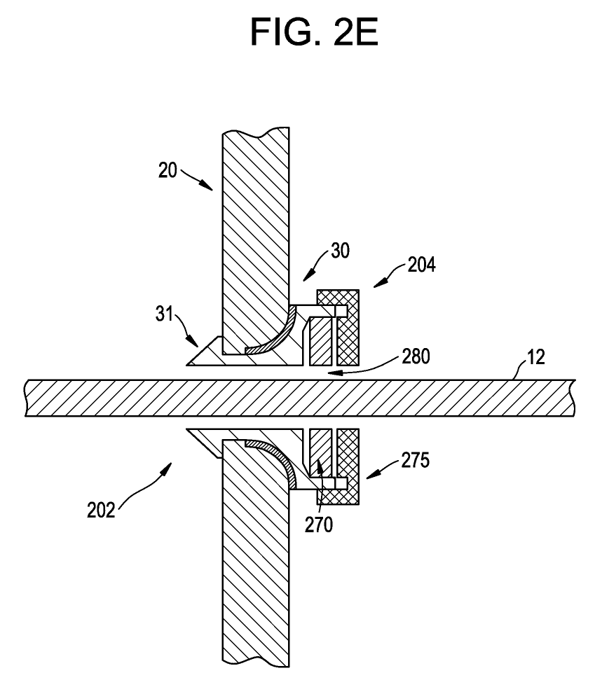

FIG. 2A illustrates a cross-sectional schematic diagram of one embodiment of a respiratory tract access port 30 implanted in a patient's airway wall 20. The respiratory tract access port 30 has a body that is formed with a channel 205 extending from its interior end 202 to its opposite exterior end 204. The interior end 202 of the body is the end that is intended for positioning interior to the airway. The exterior end 204 of the body is the end intended for positioning in the patient's thoracic cavity. It is appreciated, however, that any of the embodiments described herein may be adapted for implanting in the opposite manner, whereby the interior end 202 is positioned within the patient's thoracic cavity and the exterior end 204 is positioned within the airway.

According to various embodiments, the respiratory access port 30 has a total length between the interior end 202 and the exterior end 204 ranging from approximately 3 mm to approximately 20 mm. For example, in one embodiment, the total length is between approximately 5 mm and approximately 8 mm. However, it is appreciated that, according to other embodiments, the total length may vary.

The respiratory tract access port 30 also includes a first retaining member 210 extending from its interior end 202, and a second retaining member retaining member 215 extending from its exterior end 204 and spaced apart from the first retaining member 210. Upon implantation, the airway wall 20 is coupled between the first retaining member 210 and the second retaining member 215. According to various embodiments, the space between the first retaining member 210 and the second retaining member 215 ranges between approximately 1 mm and approximately 10 mm to accommodate the varying size of the patient's anatomy and/or the orientation of the respiratory tract access device 30. For example, in one embodiment, the space between the first retaining member 210 and the second retaining member 215 is between approximately 2 mm and approximately 6 mm. However, it is appreciated that, according to other embodiments, the amount of spacing may vary and can be adjusted by adjusting the first retaining member 210 and/or the second retaining member 215.

According to one embodiment, and as shown in FIG. 2A, the first retaining member 210 is formed in a barb-shape with a conical or semi-conical (e.g., frustoconical) end and extending radially from all or at least a portion of the surface of the respiratory tract access port 30. When formed in a barb-shape, the first retaining member 210 has a diameter that narrows along the length of the body in the direction toward the interior end 202 of the respiratory tract access port 30. The pointed configuration of the first retaining member 210 facilitates insertion through the airway wall 20, while its larger outer diameter relative to the diameter of the aperture formed in the airway wall 20 retains the respiratory tract access port 30 in place within the airway wall 20. Also as shown, the first retaining member has a face 212 that extends in an approximately perpendicular direction from the outer surface of the respiratory tract access port 30 and faces toward the exterior end 204. The face 212 is positioned to abut the surface of the airway wall 20.

According to various embodiments, the respiratory access port 30 has an outer diameter measured across the diameter of either the interior end 202 or the exterior end 204 ranging from approximately 2 mm to approximately 15 mm. For example, in one embodiment, the outer diameter is between approximately 4 mm and approximately 8 mm. However, it is appreciated that, according to other embodiments, the outer diameter may vary.

In other embodiments, the first retaining member 210 may be configured as one or more tabs extending radially from, and in an approximately perpendicular direction to, the outer surface of the respiratory tract access port 30. The one or more tabs provide the same function as the barb-shaped retaining member, abutting the surface of the airway wall 20 to retain the respiratory tract access port 30 in place.

Although the first retaining member 210 is illustrated in FIG. 2A as integral with the body of the respiratory tract access port 30, in other embodiments, the first retaining member 210 is removably attachable over the interior end 202 of the respiratory tract access port 30. The first retaining member 210 may be removably attachable using any number of attachment mechanisms, include, but not limited to, complementary threading, clips, screws, adhesive, friction fit, and the like. Accordingly, in an embodiment including a removably attachable first retaining member 210, the respiratory tract access port 30 can first be inserted through the airway wall 20, and the first retaining member 210 can then be positioned over the interior end 202 and attached to the respiratory tract access port 30, coupling the airway wall 20 between the first retaining member 210 and the second retaining member 215. In one example of this embodiment, the second retaining member 215 is integral with the respiratory tract access port 30, such that only the first retaining member is removably attachable; though, in other embodiments, both retaining members may be removably attachable or both may be integral.

According to one embodiment, the second retaining member 215 is configured as an annular lip or collar extending radially from the outer surface of the respiratory tract access port 30 at a distance spaced apart from the first retaining member 210. The second retaining member 215 is similarly configured to retain the respiratory tract access port 30 in position within the airway wall 20 due to its larger outer diameter of the first retaining member 210 relative to the diameter of the aperture formed in the airway wall 20. According to one embodiment, the second retaining member 215 is formed to slope toward the interior end 202 of the respiratory tract access port 30, graduating from a thicker cross section to a thinner cross section. The sloped portion facilitations insertion of the respiratory tract access port 30 at least partially through the airway wall 20 and also increases the external surface area of the respiratory tract access port 30 in contact with the airway wall 20, which improves the sealing function of the respiratory tract access port 30 and promotes beneficial tissue in-growth. The sloped portion further compensates for varying airway wall 20 thickness, as may occur in differing patients, applications, implantation locations, and/or tissues.

In accordance with one embodiment, the respiratory tract access port 30 is inserted into and implanted within a patient's airway by penetrating the airway wall 20 using the respiratory tract access port 30. Positioned external to the trachea 20, an axial force in a direction toward the interior of the airway is applied to the respiratory tract access port 30, forcing the respiratory tract access port 30 against the airway wall 20 and causing the first retaining member 210 to penetrate airway wall 20, forming an aperture in the trachea wall 210. The conical shape of the first retaining member 210 facilitates puncturing and increasing the aperture in the airway wall 20. Upon penetration, the respiratory tract access port 30 is positioned such that first retaining member 210 extends through and is positioned adjacent to the interior surface of the airway wall 20, and the second retaining member 215 is adjacent to the exterior surface of the airway wall 20.

In addition, according to one embodiment, the second retaining member 215, the first retaining member 210, and/or other surface areas of the respiratory tract access port 30 are covered with a porous material 217. The porous material 217 may be any porous material that promotes tissue in-growth to provide a barrier to infection and improved mechanical strength after implantation. Examples of suitable materials include, but are not limited to, Dacron or expanded polytetrafluoroethylene (ePTFE). In addition, all or part of the respiratory tract access port 30 surface can be coated with or elute various materials known in the art for promoting tissue in-growth, including, but not limited to, genes, proteins, bio-active metals, or bio-active polymers.

The respiratory tract access port 30 optionally includes means for sealing and/or mechanically constraining one or more electrical leads 12 inserted therethrough. The sealing means aid in preventing or mitigating infection of the thoracic cavity that potentially results from exposure to the airway environment. In one embodiment, the sealing means includes a lead seal 220 with a similar cross-sectional shape as the respiratory tract access port 30, and with a hollow channel defined therethrough. The lead seal 220 may be manufactured from a non-rigid elastic biocompatible material, such as, but not limited to, silicone or any other elastic biocompatible polymer.

During placement, the lead seal 220 is radially expanded to temporarily increase its inner diameter by manually exerting opposing forces from an interior channel of the lead seal 220 using reverse pliers or another suitable instrument. The lead seal 220 is then expanded and positioned at least partially over and onto the exterior end 204 of a non-implanted respiratory tract access port 30 having electrical leads 12 extending therethrough. The lead seal 220 thus seals the electrical lead 12 within the respiratory tract access port 30 for subsequent implantation into an aperture in the airway wall 20. Alternatively, the lead seal 220 is expanded over the respiratory tract access port 30 after the respiratory tract access port 30 has been implanted within the airway wall 20. The lead seal 220 can be expanded over already deployed electrical leads 12, or electrical leads 12 can be inserted through the lead seal 220. After the lead seal 220 is installed on the respiratory tract access port 30, either before, after, or during implantation, the instrument used to expand is removed and the lead seal is firmly seated on the exterior end 204 of the respiratory tract access port 30, held in place by an elastic, compressive force.

According to various embodiments, the lead seal 220 has a total length ranging from approximately 3 mm to approximately 50 mm. For example, in one embodiment, the outer diameter is between approximately 4 mm and approximately 15 mm, with at least a portion extending over the exterior end 204 of the respiratory tract access port 30 and the remaining portion extending over the electrical lead 12. However, it is appreciated that, according to other embodiments, the lead seal 220 total length may vary.

According to one embodiment, the respiratory tract access port 30 also includes a securement fitting 225 extending from at least a portion of the surface of its exterior end 204 to aid in retaining the lead seal 220 in place by engaging with or otherwise interfacing with at least a portion of an inner surface of the channel of the lead seal 220. One embodiment of a securement fitting 225, as illustrated in FIG. 2A, includes one or more barbs extending radially from the surface of the respiratory tract access port 30. In another embodiment, the securement fitting 225 is another radially extending member, such as, but not limited to, one or more lips, collars, teeth, spikes, enhanced friction surface (e.g., ridged, grooved, etched, etc.), and the like.

Because the channel of the lead seal 220 has a smaller diameter than electrical leads 12 and the fitting 225, sufficient pressure will be placed on both the electrical lead or leads 12 and the respiratory tract access port 30 to seal the interior of the airway from the thoracic cavity. In one embodiment, the hollow channel of the lead seal 220 is formed with two different inner diameters—a first smaller diameter 227 to accommodate one or more electrical leads 12, and a second larger diameter 229 to accommodate the respiratory tract access port 30. For example, according to various embodiments, the first smaller diameter 227 is between approximately 0.5 mm and approximately 5 mm (e.g., 1 mm to 3 mm in one embodiment), and the second larger diameter 229 is between approximately 1 mm and approximately 8 mm (e.g., 3 mm to 6 mm in one embodiment). However, in other embodiments, the lead seal 220 is formed with a channel diameter that is substantially the same along the length of the lead seal 220. For example, the lead seal 220 may have a single inner diameter small enough to accommodate one or more electrical leads 12, but resilient enough to stretch to accommodate the diameter of the exterior end 204 of the respiratory tract access port 30. For example, in one embodiment, the inner diameter has a small constant inner diameter ranging between approximately 1 mm and approximately 3 mm. In other embodiments, the inner diameter varies along the length of the lead seal 220, such as a gradual variation from a larger diameter to a smaller diameter. It is appreciated that the aforementioned dimensions are provided for illustrative purposes, and that the inner diameters may vary according to other embodiments.

FIG. 2B illustrates a cross-sectional schematic diagram of another embodiment of a respiratory tract access port 30 that includes a lead seal 220. According to this embodiment, the lead seal 220 is formed with one or more inner seals 222 extending radially in an inward direction from the inner surface of the channel of the lead seal to provide a seal between one or more electrical leads (not shown) and the respiratory tract access port 30. In one embodiment, the inner seals 222 are formed in an annular shape and extend from the interior surface of the lead seal 220, creating a void or orifice having a given diameter within the inner seals 222 that is smaller than the overall diameter of the hollow channel 224. In one embodiment, the orifice diameter created by the fines 222 is substantially the same or smaller than the anticipated diameter of the electrical lead or leads to be contained therein, allowing for a tight seal to be formed around the leads.

The inner seals 222 may have any number of shapes, including, but not limited to, ovular, elliptical, non-elliptical, or any other suitable shape, depending upon the intended application. In one embodiment that includes multiple electrical leads, the inner seals 222 include multiple orifices extending therethrough with each orifice accommodating one or more of the multiple electrical leads. In one embodiment, one or more sealing inner seals 222 are integrated with, or otherwise affixed to, an electrical lead instead of extending from the interior of the lead seal 220. In this embodiment, the inner seals 222 extend radially from the surface of the electrical lead and have a size (e.g., outer diameter) and shape (e.g., circular) to create a sufficient seal with the lead seal 220. In another embodiment, the inner seals 222 are formed to extend essentially entirely across the channel 224 of the lead seal 220, but include one or more slits formed therethrough for retaining one or more electrical leads. The inner seals 222 may be manufactured from a non-rigid elastic biocompatible material, such as, but not limited to silicone or any other suitable elastic biocompatible polymer.

FIG. 2C illustrates another embodiment of a respiratory tract access port 30. According to this embodiment, a removable retaining member 230 is provided as a separate component from the respiratory tract access port 30. The removable retaining member 230 can be a collar or threaded nut adapted to be positioned over the external end 204 of the respiratory tract access port 30. In one embodiment, the respiratory tract access port 30 includes threads 235 for threadably attaching threaded removable retaining member 230 having complementary threads on an interior surface. In other embodiments, however, the respiratory tract access port 30 and/or the removable retaining member 230 has any of a number of other means for securing the removable retaining member 230 to the respiratory tract access port 30, such as, but not limited to, a latch, snap, barb, friction fit, and the like.

In addition, according to one embodiment, the respiratory tract access port 30 illustrated in FIG. 2C further includes a conical (or partially conical) end 240 or otherwise substantially narrowed interior end 202 (like that described with reference to FIG. 2A), but which also includes one or more sharp-edged members 245, such as screw-like threads, helical grooves, or other sharp members or cutting implements that extend radially from the surface of the conical end 240. The sharp-edged members 245 facilitate puncturing the airway wall 20 during insertion. For example, when applying an axial pressure against the airway wall 20 with the respiratory tract access port 30, a rotating motion can also be applied, causing the sharp-edged members 245 to sever the tissue and aid penetration.

The respiratory tract access port 30 illustrated in FIG. 2C includes a channel 205 extending longitudinally along its length between the interior end 202 and the exterior end 204, which forms at least two inner diameters—a first smaller diameter 247 for securing around one or more electrical leads 12 and substantially sealing the thoracic cavity from the airway environment, and a second larger diameter 249 for more freely housing the one or more electrical leads 12. In other embodiments, however, the channel 205 may have a constant smaller diameter or a gradually varying diameter.

According to one embodiment, the respiratory tract access port 30 illustrated in FIG. 2C optionally includes a porous material 217, such as is illustrated in and described with reference to FIG. 2A, on one or more of its surfaces that will be in contact covered with the airway wall 20. As described, the porous material 217 is provided to promote tissue in-growth, to generate a barrier to infection, and/or to provide improved mechanical strength between the respiratory tract access port 30 and the airway wall 20.

FIG. 2D illustrates a respiratory tract access port 30, according to another embodiment. In this embodiment, the respiratory tract access port 30 includes an angled entry channel 250 at its exterior end 204 and an angled exit channel 255 at its interior end 202 to accommodate the orientation and direction of one or more electrical leads 12 during insertion and while implanted. In one embodiment, the angled exit channel 255 is configured to open in a distal direction toward the bronchi when implanted, thus directing an electrical lead 12 into the bronchi (or other distal portions of a patient's airway or other body lumen). The angled entry channel 250 is configured to open in the direction toward the anticipated subcutaneous implantation site for the pulse generator. The entry and exit angles of the respective angled entry channel 250 and the angled exit channel 255 may lie in the same or different planes. In one embodiment, the entire respiratory tract access port 30, or at least part of angled entry channel 250 and/or the angled exit channel 255, are formed from one or more rigid materials. Though, in other embodiments, the angled entry channel 250 and/or the angled exit channel 255 can be formed at least partially from non-rigid, semi-rigid, or pliable material, which allows adjusting the position and direction of the channels prior, during, or after implantation, as desired.

The respiratory tract access port 30 shown in FIG. 2D includes a first retaining member 210 at its interior end 202, a second retaining member 215 spaced apart from the first retaining member 210 on its exterior end 204, and a lead seal 220 slideably positioned over its exterior end 204, similar to that illustrated in and described with reference to FIG. 2A. However, in other embodiments, the respiratory tract access port 30 is configured in any of a number of other configurations, such as any of the other embodiments illustrated and/or described herein.

FIG. 2E illustrates another embodiment of a respiratory tract access port 30. It includes an inner seal 270, which may be integral with or separate from the respiratory tract access port 30, and one or more seal compression members 275, which may be a friction fit collar or a threaded nut, to facilitate sealing the respiratory tract access port 30 and one or more electrical leads 12 therein. In one example, a seal compression member 275 includes threads complementary to threads formed along at least a portion of the exterior surface of the exterior end 204 of the respiratory tract access port 30. One or more inner seals 270 are configured as a disc having a void or orifice 280 formed through the inner seal 270, such that one or more electrical leads 12 may be fed through the orifice 280. In one embodiment, the inner seal 270 is manufactured from silicone or any other suitable elastic biocompatible material. Upon threading the seal compression member 275 on the exterior end 204 of the respiratory tract access port 30, the one or more inner seals 270 are compressed axially between the seal compression member 275 and the exterior end 204, causing the diameter of the orifice 280 to be reduced and sealing the inner seal 270 around the one or more electrical leads 12.

According to one embodiment, the inner seal 270 is constructed from a pliable material and the compression member 275 and the exterior end 204 are constructed from rigid materials relative to the pliability of the inner seal 270. In this embodiment, the outer diameter of the inner seal 270 is also confined by the inner diameter of the exterior end 204. Therefore, when the compression member 275 is threaded onto the exterior end 204, the pliable inner seal 270 is compressed and the inner seal 270 inner diameter is reduced axially in the only non-constrained direction. This axial compression thus results in a reduction of the inner diameter of the inner seal 270.

FIG. 2E illustrates a distance between the orifice 280 of the inner seal 270 and the electrical lead 12 for purposes of illustration. However, upon threading the seal compression member 275, the inner seal 270 will compress the orifice 280 and contact the electrical lead 12 to create a seal therebetween.

In other embodiments, the inner seal 270 and seal compression member 275 are positioned on the interior side 202 of the respiratory tract access port 30, such that they are applied through the patient's airway. In yet other embodiments, inner seal 270 and a seal compression member 275 are positioned on both the interior side 202 and the external side 204 of the respiratory tract access port 30.

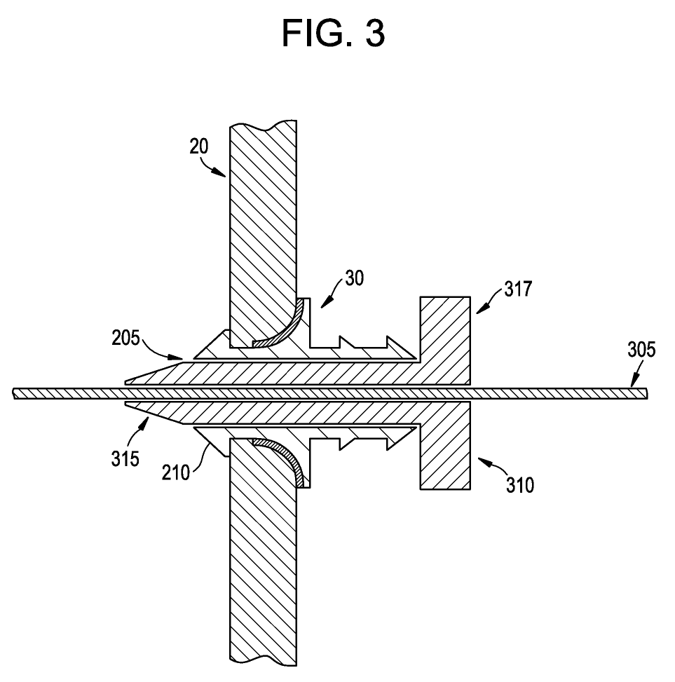

FIG. 3 illustrates a respiratory tract access port 30 and delivery apparatus used to facilitate implanting the same. According to this embodiment, a delivery apparatus includes a guidewire 305 and a dilator 310 configured to facilitate opening an aperture formed in the patient's airway wall 20 and implanting the respiratory tract access port 30 therein.

According to this embodiment, a guidewire 305 is first inserted through the airway wall 20 (or other tissue) to facilitate puncturing and penetrating the airway wall 20. The guidewire 305 may be inserted in any known manner, such as by performing the Seldinger technique, or any other suitable technique.

A dilator 310 is adapted to slide over the guidewire 305 and to expand the aperture formed in the airway wall 20 when inserted therethrough. According to one embodiment, the dilator is adapted to fit within at least a portion of the channel 205 of the respiratory tract access port 30 to further assist expanding the aperture in the airway wall 20 and inserting the respiratory tract access port 30. For example, the interior end 315 of the dilator may be configured in a conical or partially conical shape (e.g., frustoconical), narrowing to a smaller outer diameter than the inner diameter of the channel 205 of the corresponding respiratory tract access port 30.

According to one embodiment, the guidewire 305 is first inserted through the trachea wall 20 creating an aperture therein. After inserting the guidewire 305, the dilator 310 can be inserted over the guidewire 305 (from within the thoracic cavity or external to the patient for insertion through the thoracic cavity), with the interior end 315 pointing toward the airway wall 20. The dilator 310 is then inserted into the aperture of airway wall 20 that was initially created by the guidewire 305, gradually increasing its diameter. In one embodiment, the dilator 310 is already positioned within the channel 205 of the respiratory tract access port 30, such that both the dilator 310 and the respiratory tract access port 30 will be pushed together through the aperture in the airway wall 20. In one embodiment, the dilator includes a foot 317 that extends radially from its external end, which serves to abut the external end of the respiratory access port 30 and cause the respiratory access port 30 to be pushed by the dilator 310. In other embodiments, however, the respiratory tract access port 30 is passed over the guidewire 305 and over the dilator 310 after their insertion. In these embodiments, the shape of the dilator 310 will be modified from that illustrated in FIG. 3 to not include the foot 317. After fully implanting the respiratory tract access port 30 into the airway wall 20, the dilator 310 and the guidewire 305 are removed.

In some embodiments, only the guidewire 305 or only the dilator 310 are used to form and/or increase the aperture in the airway wall 20 and to facilitate insertion the respiratory tract access port 30 therein. It is further appreciated that, according to other embodiments, the orientation of the dilator 310 may be reversed, permitting inserting the respiratory access port 30 from within the airway, through the airway wall 20, and into the thoracic cavity in reverse orientation.

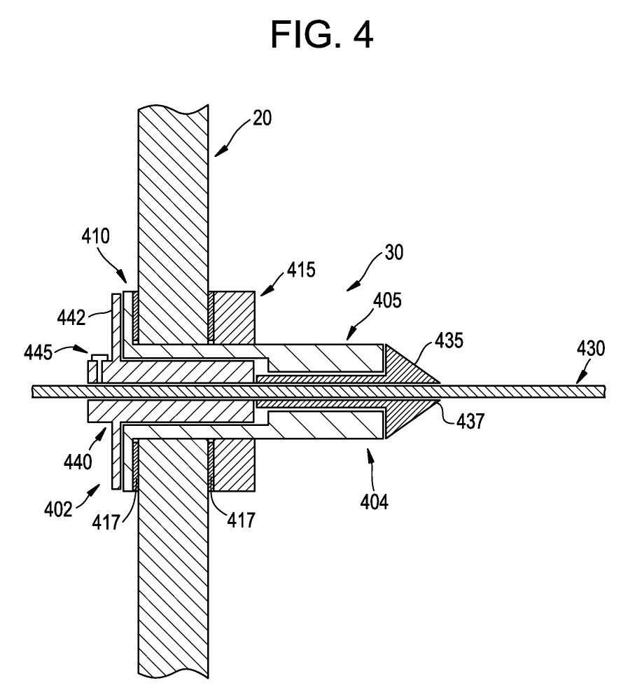

FIG. 4 illustrates another embodiment of a respiratory tract access port 30 and a delivery apparatus. The respiratory tract access port 30 of this embodiment is implantable in a patient's airway wall 20 from the airway side. According to this embodiment, the respiratory tract access port 30 has a body with an external seal portion 405, an internal retaining member 410, and an external retaining member 415. The external seal portion 405 extends through the airway wall 20 into the patient's thoracic cavity. The internal retaining member 410 is positioned adjacent to the interior surface of the airway wall 20. As shown in FIG. 4, the internal retaining member 410 can be integral with the body of the respiratory tract access port 30, or it may be removably attachable (e.g., threaded, friction fit, tabs, etc.). The external retaining member 415 is threadably attachable (or attachable by any other suitable means, such as friction fit, tabs, etc.) over the external seal portion 405. The external retaining member 415 secures the respiratory tract access port 30 against the airway wall 20.

The respiratory tract access port 30 also optionally may include a lead seal positioned over external seal portion 405 of the external end 404 and/or over the internal end 402 of the respiratory tract access port 30. The lead seal may be configured in any manner, such as similar to the embodiments illustrated in and described with reference to FIGS. 2A-2E.

In one embodiment, the respiratory tract access port 30, internal retaining member 410, the external seal portion 405, the lead seal, and/or the external retaining member 415, or any portions thereof, are manufactured from any suitable non-rigid, elastic biocompatible material, such as silicone. The respiratory tract access port 30, the internal retaining member 410, and/or the external retaining member 415, or any portions thereof, also optionally may include a porous material that promotes tissue in-growth to provide a barrier to infection and improved mechanical strength, such as the porous material 217 described with reference to FIG. 2A. Various portions of the respiratory tract access port 30 also optionally may be coated with or elute various materials known in the art for promoting tissue in-growth, including, but not limited to, genes, proteins, bio-active metals, or bio-active polymers.

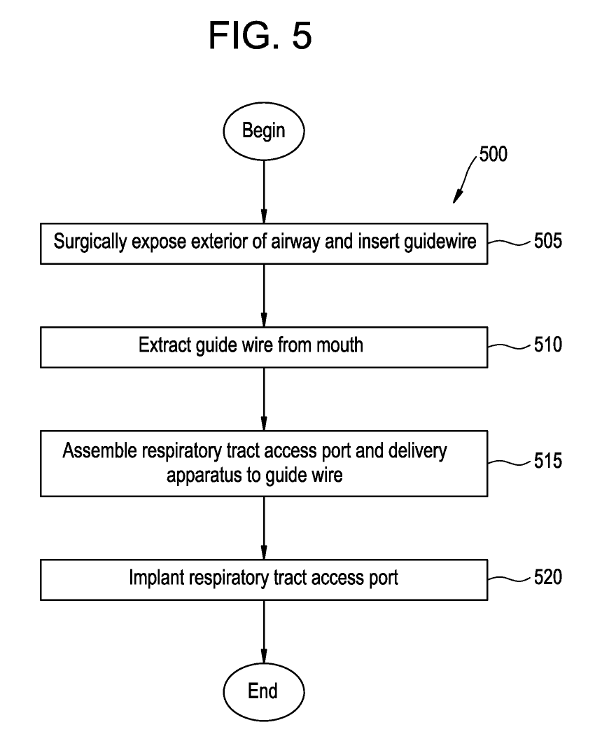

In the embodiments illustrated in FIG. 4, the respiratory tract access port 30 can be implanted using a guidewire 430 and a dilator 435. As shown in FIG. 4, the dilator 435 optionally may include a pull plate 440 having an integrated set screw 445 (or other securing means) for removably attaching the pull plate to the guidewire 430. The pull plate 440 also may include a radially extending foot 442, which serves to abut the internal retaining member 410 and cause the respiratory access port 30 to be pulled by the dilator 435 when pulling the guidewire 430. Methods for implanting the respiratory tract access port 30 using a guidewire 430 and a dilator 435 is further described with reference to FIG. 5 below.

Although FIG. 4, and the corresponding method of implanting described with reference to FIG. 5 below, illustrate this embodiment of the respiratory tract access port 30 as being implantable from within a patient's airway, in other embodiments, the respiratory tract access port 30 is adaptable for implantation from a subcutaneous position, such as from a patient's thoracic cavity. In such embodiments, the orientation of the respiratory tract access port 30 would be reversed from that illustrated in FIG. 4, with the pointed tip of the dilator 435 oriented toward the interior of the airway.

FIG. 5 illustrates a method 500 for implanting a respiratory tract access port 30, according to the embodiment illustrated in and described with reference to FIG. 4 and in which the respiratory tract access port 30 is implanted from the patient's airway.

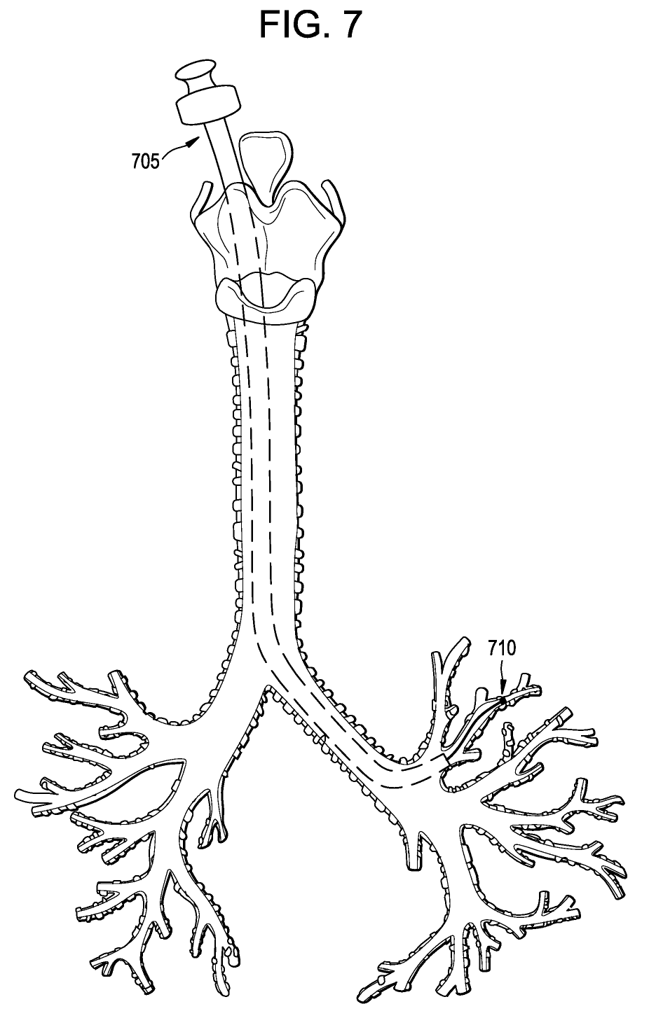

The method 500 begins at block 505, in which a guidewire 430, as described with reference to FIG. 4, is inserted into the airway from the patient's thoracic cavity and is used to penetrate the airway wall 20. In another embodiment, however, the guidewire 430 is inserted through the patient's mouth or nasal cavity and through the airway to the desired site, penetrating the airway wall from the airway side and forming an aperture therein, and deploying the guidewire 430 into the thoracic cavity. In one embodiment, the Seldinger technique or other similar technique is used to puncture the airway wall and deploy the guidewire 430. In another embodiment, the airway is exposed surgically to permit better access thereto. Moreover, as described with more detail with reference to FIG. 7, an endoscope may be used to locate and facilitate deploying the guidewire 430.

Following block 505 is block 510, in which the guidewire 430 is advanced and extracted from the patient's mouth (or nasal cavity). The guidewire 430 can be extracted with the aid of forceps, catheters, endoscopes, and/or any other suitable instrument for extending and grasping within a lumen. With the guidewire 430 extending through the airway wall 20 and passing out of the patient's mouth, the respiratory tract access port 30 can then be positioned over the guidewire 430 and deployed to and positioned within the aperture previously formed in the airway wall 20 at block 505, as described below.

Block 515 follows block 510, in which the respiratory tract access port 30 is optionally assembled on a dilator 435, such as is described with reference to FIG. 4, outside of the patient's airway or within the mouth. According to one embodiment, the dilator 435 is a two-piece dilator that includes a needle tip 437 and a pull plate 440. The pull plate 440 is optionally removably attached to the guidewire 430 by set screw 445, or any other suitable attachment means, to facilitate pulling the assembled respiratory tract access port 30 and dilator 435 by the guidewire 430. According to one embodiment, the pull plate 440 includes a radially extending foot 442, which may be configured in an annular- or collar-shape, that is positioned to abut and, thus, drag along the respiratory tract access port 30 with the guidewire 430. In other embodiments, the pull plate 440 includes one or more members for abutting and engaging the respiratory tract access port 30 that are not shaped like a collar, such as one or more extending tabs, for example.

Following block 515 is block 520, in which the respiratory tract access port 30 is implanted in the aperture formed in the airway wall 20 at block 505. In one embodiment, the guidewire 430 is pulled from the thoracic cavity through the aperture formed in the airway wall 20 until the needle tip 437 of the attached dilator 435 further expands the aperture and exits from the airway and into the thoracic cavity. The dilator 435 is pulled until the internal retaining member 410 of the respiratory tract access port 30 is adjacent to and in contact with the interior wall of the airway.

Upon suitable positioning of the respiratory tract access port 30, an external retaining member 415, such as is described with reference to FIG. 4, is positioned over the guidewire 430 from the thoracic cavity, over the needle tip 437 of the dilator 435 until the external retaining member 415 contacts the exterior wall of the airway. In one embodiment, the external retaining member 415 is non-rigid and forms an orifice with an inner diameter smaller than the outer diameter of the respiratory tract access port 30, which allows the external retaining member 415 to be retained on the respiratory tract access port 30 by a friction fit. In another embodiment, the external retaining member 415 is attached to the respiratory tract access port 30 by one of any other suitable means. For example, the external retaining member 415 and at least a portion of the outer surface of the respiratory tract access port 30 may include complementary threads for threadably attaching the external retaining member 415 to the respiratory tract access port 30. Upon positioning and securing the respiratory tract access port 30 by securing the external retaining member 415 and the internal flange 410 against the airway walls, the dilator 435, the pull plate 440, and the guidewire 430 are withdrawn. Some or all of these components may be removed from the patient's mouth or nasal cavity and/or from the patient's thoracic cavity.

While FIG. 5 illustrates one embodiment of deploying and implanting a respiratory tract access port 30 from within a patient's airway, other embodiments may include deploying some or all of the respiratory tract access port 30 components and delivery devices from the thoracic cavity or from any other means of accessing the desired implantation site. Moreover, according to other embodiments, similar systems and methods can be used to deploy and implant cannula or other access ports in other tissues, such as within a patient's digestive tract, or a combination of a patient's digestive tract and airway creating an access port through both.

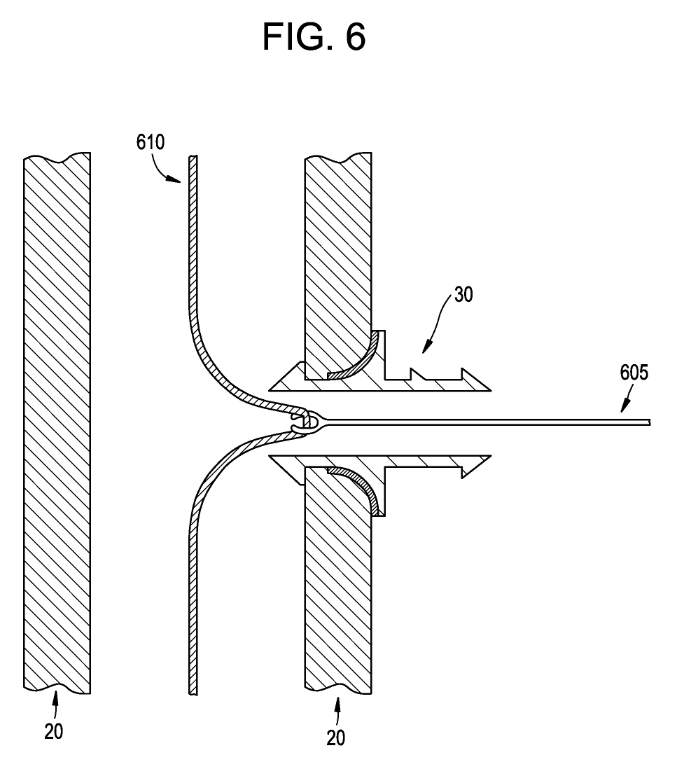

FIG. 6 illustrates an apparatus used to extract a guidewire and/or electrical lead from a patient's airway (or other body lumen) through a respiratory tract access port, or any other cannula implanted within or orifice created through a tissue wall. These apparatus and corresponding methods may be used to deploy electrical leads for attachment to a subcutaneously implanted pulse generator, for retrieving electrical leads from the thoracic cavity and deploying to the patient's airway, or for positioning a guidewire to aid implantation of other devices.

According to one embodiment, a grasping instrument 605, such as, but not limited to, forceps, a lasso, or a snare, is inserted through the respiratory tract access port 30 from the thoracic cavity and into the airway. The grasping instrument 605 is used to grasp one or more electrical leads 610 (or guidewire) and to extract the electrical lead 610 through the respiratory tract access port 30 and into the thoracic cavity.

In another example, the grasping instrument 605 is used to grasp a guidewire. According to this embodiment, the guidewire can then be used to deploy and position any other devices into and/or through the respiratory tract. For example, the guidewire can facilitate positioning one end of an electrical lead 610 in the airway and the other end to a pulse generator contained within the patient's thoracic cavity.

In another embodiment, an access port guidewire that is integrated with and/or used to implant a respiratory tract access port 30 (such as the guidewire 430 illustrated in and described with reference to FIGS. 4-5) is used to extract the connector end of an electrical lead 610 from the patient's mouth (or nasal cavity) or to extract an additional guidewire. For example, the end of the access port guidewire 430 positioned within a patient's airway or extending outside of the patient's mouth or nasal cavity, which was initially used to implant the respiratory tract access port 30, is connected to the electrical lead 610 (or to an another guidewire). By retracting the guidewire 430 from the airway through the respiratory tract access port 30, the attached electrical lead 610 (or additional guidewire) will also be extracted from the airway, through the respiratory tract access port 30, and into the thoracic cavity.

FIG. 7 illustrates one embodiment of a system used to facilitate navigating to the general vicinity of a desired implantation and/or stimulation site. In one embodiment, an endoscope 705, such as a bronchoscope, is used to deploy components, such as guidewires, dilators, respiratory tract access ports, electrical leads carrying one or more electrodes, and the like, to a desired site. According to some embodiments, other suitable navigation devices and techniques including, but not limited to, fluoroscopy, computed tomography, magnetic resonance imaging, x-ray, ultrasound, or position emission tomography also are used to facilitate guidance and deploying of one or more components.

In one embodiment, an endoscope 705 that includes a working channel is used. A temporary wire 710 is inserted through the working channel of the endoscope 705 to the desired implantation or stimulation site. According to one embodiment, upon positioning the temporary wire 710, a stimulation signal and/or sensing signal is delivered via the temporary wire 710 (or any other electrical lead) to the stimulation site from a pulse generator or other controller to identify the desired location of the implantation or stimulation site. In one embodiment, upon finding a desired suitable location, the endoscope 705 is removed, leaving the temporary wire 710 in place as a marker. In another embodiment, however, the temporary wire 710 is replaced by a different wire, such as a thinner marking wire, prior to removing the endoscope 705. One or more electrical leads are then deployed to the desired location over the temporary wire 710 or over any other different marking wire or guidewire.

According to another embodiment, a catheter is positioned over the temporary wire 710, and one or more electrical leads are deployed via an internal channel of the catheter. In another embodiment, however, a catheter can be positioned over the endoscope 705 prior to its insertion into the airway, leaving the catheter in position when the endoscope 705 and/or the temporary wire 710 is removed. In yet another embodiment, one or more electrical leads are guided to the desired stimulation site through the working channel of the endoscope 705 prior to removal of the bronchoscope, thus avoiding the need to use a temporary wire 710 or any other marking wire.

In the embodiment illustrated in FIG. 7, the endoscope 705 is inserted into the airway through the patient's mouth or nasal cavity. In another embodiment, the endoscope 705 is inserted into the patient's airway from the patient's thoracic cavity through a previously implanted respiratory tract access port, such as any respiratory tract access port 30 described with reference to FIGS. 2-5.

According to one aspect, a method for treating a patient is provided that includes the deployment and implantation of any of the access port embodiments described herein with reference to FIGS. 1-7 in a tissue wall. As part of the method for treating a patient, one or more electrical leads each carrying one or more stimulation and/or sensing electrodes are deployed and contained within the access port. A pulse generator or other controller is also deployed and implanted within the patient. After implantation, and optional testing of the electrical leads and electrodes for location and/or operation, one or more stimulation and/or sensing signals are delivered from the pulse generator via the one or more electrical leads contained within the access port. A further aspect of treatment can include safe removal of the access port and other device components from the patient.

According to another aspect of the invention, an access port implantation kit is provided that includes one or more of the components described herein with reference to FIGS. 1-7. An access port implantation kit can be packaged for individual use during an implantation procedure of an access port, with any other implantable devices, such as a cardiac, diaphragm, or phrenic nerve stimulator, and/or with any other delivery devices, such as an endoscope. For example, according to one embodiment, an access port implantation kit includes at least an access port, a guidewire, and a dilator. The access port, guidewire, and dilator may be configured according to any of the embodiments described with reference to FIGS. 2-6. In one embodiment, multiple different lead seals or other sealing members described herein can be included in the access port implantation kit, allowing affixing different lead seals in different configurations as desired. It is appreciated that an access port implantation kit may be designed with an access port and corresponding components of a pre-determined size, and that multiple different sized kits can be available, depending upon the intended use and implantation site. In some embodiments, the access port implantation kit can further include one or more electrical leads either already positioned within the access port or for insertion during or after implantation of the access port. Similarly, the access port implantation kit can further include a pulse generator or other controller for transmitting and/or receiving stimulation and/or sensing signals or commands. In one embodiment, a grasping instrument for grasping and pulling an electrical lead, guidewire, or any other device through the access port, as illustrated in and described with reference to FIG. 6, is also included in the access port implantation kit.

Accordingly, the devices and associated methods described herein facilitate deployment and containment of electrical leads that pass through a patient's tissue wall. The example access ports described herein can be for implantation into and through any tissue wall, and are not intended to be limited to an airway wall. Containing one or more electrical leads within an access port implanted through a tissue wall serves to reduce mechanical fatigue on the electrical leads. The access ports further serve to reduce irritation of the patient's tissue wall, which would otherwise result from leads passing directly through the tissue wall without the use of an access port. Finally, the example sealing features of the access ports described herein further provide a barrier between the different biological environments that exist on different sides of a tissue wall (e.g., sealing the airway from the thoracic cavity or the gastrointestinal tract from the thoracic cavity), which further avoids infection during and after implantation of electrical leads. As a result, these access ports increase the effectiveness and safety of new cardiac stimulation devices and techniques that entail passing electrical leads through a patient's tissue wall, such as those requiring electrical leads passing from the patient's thoracic cavity and into the patient's airway for tissue stimulation from within the airway.

Publications cited herein are incorporated by reference. Modifications and variations of the methods and devices described herein will be obvious to those skilled in the art from the foregoing detailed description. Such modifications and variations are intended to come within the scope of the appended claims.

We claim:

1. An implantable port device for providing access through a tissue wall of a lumen of a patient's body, comprising:
a body comprising a first end having a first opening and an opposed second end having a second opening, and a channel extending from between and operably connecting the first opening and the second opening;
a first retaining member extending radially from the first end of the body; and
a second retaining member spaced apart from the first retaining member, the second retaining member being closer than the first retaining member to the second end of the body, and extending radially from the second end of the body,
wherein the first retaining member and the second retaining member are configured to cooperatively engage opposing sides of the tissue wall about edges of an aperture through the tissue wall to secure the body within the aperture.

2. The device of claim 1, wherein the first retaining member is tapered toward the first end of the body.

3. The device of claim 1, wherein the first retaining member has a face approximately perpendicular to the outer surface of the body, wherein the face is oriented toward the second end of the body.

4. The device of claim 1, wherein the first retaining member comprises one or more tabs extending radially from, and in an approximately perpendicular direction to, the outer surface of the body.

5. The device of claim 1, wherein the first retaining member is fixed to the first end of the body.

6. The device of claim 1, wherein the first retaining member is removably attachable to the first end of the body.

7. The device of claim 1, wherein the tissue wall comprises a trachea, a bronchus, or a digestive tract.

8. The device of claim 1, wherein the second retaining member comprises a collar adapted to slide over the second end of the body.

9. The device of claim 1, wherein the second retaining member is adapted to threadably engage at least a portion of the second end of the body.

10. The device of claim 1, wherein the second retaining member is adapted to engage at least a portion of the second end of the body at least partially by friction between an inner surface of the second retaining member and an outer surface of the second end of the body.

11. The device of claim 1, further comprising a lead seal comprising a channel, wherein the channel of the lead seal is positionable over at least a portion of the second end of the body.

12. The device of claim 11, wherein the channel of the lead seal has a first inner diameter and a second inner diameter, the first inner diameter being smaller than the outer diameter of the second end of the body, and the second inner diameter being smaller than the first inner diameter.

13. The device of claim 11, wherein the lead seal comprises an elastic biocompatible material.

14. The device of claim 11, wherein the body further comprises one or more lead seal securement fittings extending from at least a portion of the outer surface of the second end of the body, the securement fittings interfacing with at least a portion of the inner surface of the channel of the lead seal.

15. The device of claim 14, wherein the one or more securement fittings are selected from the group consisting of barbs, teeth, spikes, ridges, grooves, collars, etched surface, and any combinations thereof.

16. The device of claim 11, wherein the lead seal further comprises an inner seal extending radially in an inward direction from the inner surface of the channel of the lead seal.

17. The device of claim 16, wherein the inner seal has an annular shape.

18. The device of claim 16, wherein the inner seal is substantially disc-shaped and comprises one or more slits extending therethrough.

19. The device of claim 18, further comprising a seal compression member adapted to engage the second end of the body over the inner seal, and to cause at least partial inward radial compression of the inner seal.

20. The device of claim 1, wherein the at least one member extending radially from the first end of the body further comprises one or more sharp-edged members extending therefrom, wherein the one or more sharp-edged members facilitate forming an aperture in the tissue wall during insertion of the first end of the body.

21. The device of claim 1, wherein at least one of the first end of the body or the second end of the body forms an angled channel, wherein the each angled channel facilitates placement of one or more electrical leads extending through the channel of the body.

22. The device of claim 1, further comprising:
a guidewire adapted to penetrate the tissue wall to form an aperture therein; and
a removable dilator adapted to slide over the guidewire and to expand the aperture when inserted therethrough.

23. The device of claim 22, wherein the dilator is adapted to be removably affixed the guidewire, wherein pulling the guidewire pulls the dilator when affixed thereto.

24. The device of claim 22, wherein the dilator is adapted to fit within at least a portion of the channel of the body, and wherein inserting the dilator through the aperture formed in the tissue wall facilitates insertion of the first end of the body positioned thereover.

25. The device of the claim 22, wherein the dilator comprises an at least partially conical end adapted for insertion through the aperture formed in the tissue wall, and wherein the conical end of the dilator narrows to a diameter more narrow than the inner diameter of the channel of the body at the first end.

26. A method of implanting an access port device in a patient in need thereof, comprising:
penetrating a lumenal tissue wall using a guidewire, forming an aperture therein;
attaching an access port device to the guidewire;
pulling the guidewire through the tissue wall in a manner effective to pull the access port into a position within the aperture of the tissue wall;
detaching the guidewire from the access port device; and
removing the guidewire from the lumen of the lumenal tissue wall.

27. The method of claim 26, wherein the guidewire and the access port are inserted into the tissue wall from the thoracic cavity.

28. The method of claim 26, wherein the guidewire and the access port are inserted into the tissue wall from the lumen of the lumenal tissue wall.

29. The method of claim 26, further comprising removably attaching a dilator in front of or integrated with the access port prior to pulling the guidewire.

30. The method of claim 26, further comprising removably attaching a pull plate to the access port device, wherein attaching the guidewire to the access port device comprises attaching the guidewire to the pull plate.

31. The method of claim 26, further comprising deploying at least one electrical lead carrying at least one electrode through a channel in the access port device, wherein the at least one electrical lead is inserted from within the lumen into the thoracic cavity or inserted from the thoracic cavity into the lumen.

32. The method of claim 26, further comprising:
after positioning the access port device in the tissue wall, grasping at least one of an electrical lead or a guidewire using a grasping instrument; and
pulling said grasping instrument causing the guidewire or the electrical lead to pass through said access port device.

33. The method of claim 26, further comprising:
accessing the tissue wall using an endoscope; and
deploying the guidewire to the tissue wall through a working channel of the endoscope.

34. A kit for implanting an access port device in a tissue wall of a lumen of a patient's body, comprising:
an access port device comprising:
a body comprising a first end having a first opening and an opposed second end having a second opening, and a channel extending from between and operably connecting the first opening and the second opening;
a first retaining member extending radially from the first end of the body; and
a second retaining member spaced apart from the first retaining member, the second retaining member being closer than the first retaining member to the second end of the body, and extending radially from the second end of the body;
a guidewire for penetrating the tissue wall and forming an aperture therein, and/or for inserting the access port device through the aperture formed in the tissue wall; and
a dilator for enlarging the aperture formed in the tissue wall.
